(19) United States
(12) Patent Application Publication
(10) Pub. No.: US 2005/0065607 A1
(43) Pub. Date: Mar. 24, 2005

(54) Assembled fusion implant

(76) Inventor: Jeffrey M. Gross, Memphis, TN (US)

Correspondence Address: Woodard, Emhardt, Moriarty, McNett & Henry LLP;Bank One Center/Tower, Suite 3700, 111 Monument Circle, Indianapolis, IN, 46204-5137, US

(21) Appl. No.: 10/669,779

(22) Filed: Sep. 24, 2003

(51) Int. Cl. A61F 2/44; A61F 2/28

(52) U.S. Cl. 623/17.11; 623/23.63

(57) ABSTRACT

A fusion implant includes a plurality of bone pieces joined together by pinning.

### BACKGROUND

Implants for use in fusing adjacent bony structures facilitate fusion by maintaining the adjacent bony structures in a predetermined spaced relationship while bone grows between them. In some cases these implants are formed from body tissues. In forming a fusion implant from body tissue, a source of tissue, such as a bone, is formed into pieces meeting the desired shape and strength requirements for a particular implant. In the case of bone, the requirements are often specified in terms of a minimum wall thickness, minimum load bearing capacity, and/or geometric size and shape. A portion of the source tissue, including pieces removed in forming implants, will fall short of the requirements to form an integral implant. Thus, it is often difficult to obtain a high yield from a particular source.

### SUMMARY

The present invention provides a fusion implant for use between adjacent bony structures, for example, such as to facilitate fusion of the bony structures.

In one aspect of the invention, a fusion implant for insertion between opposing bony structures in load bearing arrangement comprises a body having first and second pieces of bone assembled together to form a construct having exterior surfaces; and at least one pin projecting into the first and second body pieces to hold them together, the pin having a first end and a second end, a portion of the pin tapering between the first and second ends.

In another aspect of the invention, a fusion implant for insertion between opposing bony structures in load bearing arrangement comprises a body having first and second pieces of bone assembled together to form a construct; and at least one pin projecting into the first and second body pieces to hold them together, the pin having a first dimension adjacent an end and a second dimension spaced from the end into the body.

In another aspect of the invention, a fusion implant for insertion between opposing bony structures in load bearing arrangement comprises a body having first and second pieces of bone assembled together to form a construct having exterior surfaces; and a pin projecting into the first and second body pieces to hold them together, the pin angling through the construct obliquely such that it is neither parallel nor perpendicular to any of the exterior surfaces.

In another aspect of the invention, a fusion implant for insertion between opposing bony structures in load bearing arrangement comprises a body having first and second pieces of bone assembled together; and a pin projecting into the first and second body pieces to hold them together, the pin being embedded within the body, such that it is surrounded on all sides by the body.

In another aspect of the invention, a fusion implant for insertion between opposing bony structures in load bearing arrangement comprises a body having first, second, and third pieces of bone assembled together, the pieces being aligned side-by-side with the second piece of bone positioned between the first and third pieces of bone such that each piece spans the adjacent bone structures, the body including an opening through the third piece communicating with the adjacent bony structures.

In another aspect of the invention, a fusion implant for insertion between opposing bony structures in load bearing arrangement comprises a body having first, second, third and fourth pieces of bone assembled together, the pieces being aligned side-by-side with the second and third pieces of bone positioned between the first and fourth pieces of bone such that each piece spans the adjacent bony structures, the second and third pieces being spaced apart to form an opening through the body communicating with the adjacent bony structures.

In another aspect of the invention, a system for use in fusing adjacent bony structures, comprises a body having first and second pieces of bone assembled together to form a construct having exterior surfaces; a pin projecting into the first and second body pieces to hold them together, the pin having a first end and a second end, the portion of the pin tapering between the first and second ends; and a fixation device attachable to the adjacent bony structures and having a structure to limit relative motion between the adjacent bony structures.

In another aspect of the invention, a system for use in fusing adjacent bony structures, comprises a body having first and second pieces of bone assembled together to form a construct having exterior surfaces; a pin projecting into the first and second body pieces to hold them together, the pin angling through the construct obliquely such that it is neither parallel nor perpendicular to any of the exterior surfaces; and a fixation device attachable to the adjacent bony structures and having a structure to limit relative motion between the adjacent bony structures.

In another aspect of the invention, a system for use in fusing adjacent bony structures, comprises a body having first, second, and third pieces of bone assembled together, the pieces being aligned side-by-side with the second piece of bone positioned between the first and third pieces of bone such that each piece spans the adjacent bone structures, the body including an opening through the third piece communicating with the adjacent bony structures; and a fixation device attachable to the adjacent bony structures and having a structure to limit relative motion between the adjacent bony structures.

In another aspect of the invention, a method of treating adjacent bony structures comprises providing a body having first and second pieces of bone assembled together to form a construct having exterior surfaces, and a pin projecting into the first and second body pieces to hold them together, the pin having a first end and a second end, a portion of the pin tapering between the first and second ends; and positioning the implant between the adjacent bony structures in load bearing arrangement.

In another aspect of the invention, a method of treating adjacent bony structures comprises providing a body having first and second pieces of bone assembled together to form a construct having exterior surfaces, and a pin projecting into the first and second body pieces to hold them together, the pin angling through the construct obliquely such that it is neither parallel nor perpendicular to any of the exterior surfaces; and positioning the implant between the adjacent bony structures in load bearing arrangement.

In another aspect of the invention, a method of treating adjacent bony structures comprises providing a body having first, second, and third pieces of bone assembled together, the pieces being aligned side-by-side with the second piece of bone positioned between the first and third pieces of bone such that each piece spans the adjacent bone structures, the body including an opening through the third piece communicating with the adjacent bony structures; and positioning the implant between the adjacent bony structures in load bearing arrangement.

In another aspect of the invention, a method of making a bone implant comprises forming first and second bone pieces; assembling the first and second pieces to form a body; and inserting a pin into the body to hold the assembly together, the pin having a first end and a second end, a portion of the pin tapering between the first and second ends. In another aspect of the invention, a method of making a bone implant comprises forming first and second bone pieces; assembling the first and second pieces to form a body having exterior surfaces; and inserting a pin into the body to hold the assembly together, the pin angling through the construct obliquely such that it is neither parallel nor perpendicular to any of the exterior surfaces.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a perspective view of an illustrative implant according to the present invention.

FIG. 2 is a front elevation view of the implant of FIG. 1.

FIG. 3 is a top plan view of the implant of FIG. 1.

FIG. 4 is a side elevation view of the pins used in the implant of FIG. 1.

FIG. 5 is a side elevation view of the implant of FIG. 1 as part of a bone fusion system including an optional fixation device.

### DETAILED DESCRIPTION

Embodiments of a fusion implant include a body for placement between adjacent bony structures. The body comprises an assembly of pieces of bone held together with one or more pins. The adjacent bony structures may include vertebrae, long bones, and cranial bones, among others.

The body has exterior surfaces defining the shape of the implant. Some of the exterior surfaces contact the adjacent bony structures to maintain the bony structures in a desired spaced relationship during healing or fusion. The body may provide structural support up to the limits of its load bearing capacity. The body may be shaped to fill some or all of the space between the adjacent bony structures. The body may have any suitable shape including rectangular prism, sphere, hemisphere, cone, cylinder, and other suitable shapes and combinations of shapes. The body may include cancellous bone, cortical bone, uni-cortical bone, bi-cortical bone, tri-cortical bone, demineralized bone, partially demineralized bone, and/or other suitable materials. For example, the body may comprise pieces of cortical and/or cancellous bone assembled together to meet geometric size and strength requirements.

The pins used to secure the assembly may have a first dimension adjacent an end and a second dimension spaced from the end different from the first dimension to facilitate tight engagement of the pins with the bone pieces. For example, a rectangular pin may have first and second heights. Similarly, a pin may have first and second areas. In the case of a round pin it may have first and second diameters. The pin may taper between the two dimensions over a portion of their length. The pins may go all the way through the assembly so that they are exposed at two ends, part-way through the assembly so that they are exposed at one end, First Named Inventor: Jeffrey M. Gross or may be totally embedded within the assembly. The pins may be placed obliquely such that they are neither parallel nor perpendicular to the outside surfaces of the implant in order to increase the resistance of the pieces to disassembly.

The pins may have a variety of shapes. For example, they may have a cross sectional shape that is round, rectangular, “I”-shaped, “T”-shaped, “C”-shaped or other suitable shape. They may be cylindrical, rectangular, tapered, or other suitable longitudinal shape. The pins may be made from bone, metal, ceramic, carbon, bioglass, and/or polymers and combinations thereof. If bone, the pins may comprise cancellous bone, cortical bone, and combinations of cancellous and cortical bone. Further, the bone may be mineralized, partially demineralized, or fully demineralized. If the pins include polymers, they may be resorbable or non-resorbable and include polyethylene, polyester, polyglycolic acid, polylactic acid, polyaryletherketone, polyetheretherketone, polytetrafluroethylene, and/or other suitable polymers and combinations.

Bone for an assembled fusion implant may be obtained from any suitable bone source including the implant recipient as in an autograft, another source of the same species as in an allograft, or a source of a different species as in a xenograft. Suitable examples of musculoskeletal tissue include ilium, humerus, tibia, femur, fibula, patella, ulna, radius, rib, vertebral bodies, and/or other suitable bones. The bone pieces may be machined, cut, planed, and/or otherwise removed and/or formed from the donor bone.

The body may include one or more openings to facilitate fusion of the adjacent bony structures. The body may include a material to promote fusion of the adjacent bony structures incorporated into the body itself or placed in the openings formed in the body. Such bone growth-promoting material may include bone paste, cancellous bone, bone chips, bone morphogenic protein (BMP), LIM mineralization protein (LMP), platelet derived growth factors, bone marrow aspirate, stem cells, biologic growth factors, and/or other suitable materials and combinations thereof.

Combining bone pieces into a fusion implant allows the use of bone pieces having less than a predetermined minimum load bearing capacity and/or a predetermined geometry outside of a predetermined standard. The combination forms an assembled load-bearing implant that achieves the predetermined capacity and/or geometry.

The implant may be used in conjunction with a fixation device to form a bone fixation system. The fixation device may be attached to the adjacent bony structures to limit the relative motion between them. The fixation device may substantially prevent all relative motion, or it may allow a predetermined amount of motion during the healing and fusion processes.

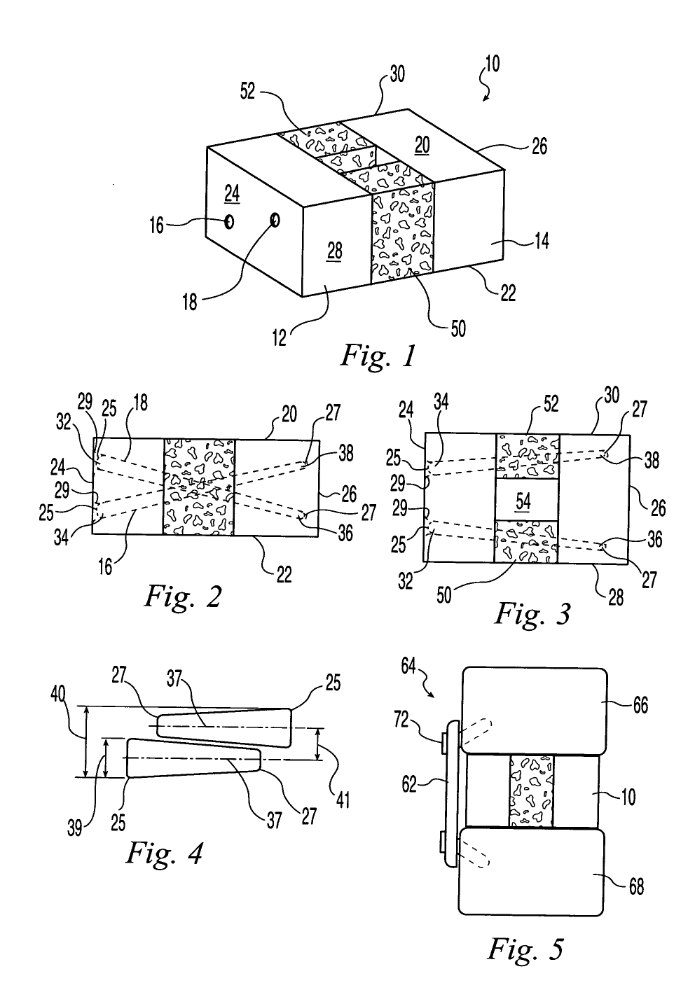

Referring to FIGS. 1-3, an illustrative embodiment of a fusion implant 10 includes first 12 and second 14 bone pieces held together with first 16 and second 18 bone pins. By using two pins 16,18 the bone pieces 12,14 are prevented from rotating relative to one another. The assembled implant is in the form of a rectangular prism having six exterior surfaces 20, 22, 24, 26, 28, 30. As seen in FIGS. 2 and 3, the pins are positioned obliquely so that they are neither parallel nor perpendicular to any of the exterior surfaces. In FIG. 2 it can be seen that the pins are oblique relative to four of the exterior surfaces 20, 22, 24, 26 and in FIG. 3 it can be seen that the pins are also oblique to the other two 28, 30 exterior surfaces. The first pin 16 angles into the body upwardly from the bottom 22 and outwardly toward one side 30. The second pin 18 angles downwardly and outwardly away from the first pin 16. The pins 16, 18 taper from a first end 32, 34 having a first diameter to a second end 36, 38 having a second diameter. The pins 16, 18 in the example are pressed into the assembly so that they wedge tightly into place. The pins 16, 18 are shown with their first ends 32, 34 adjacent an exterior surface 32 of the implant and their second ends 36, 38 buried within the implant. However, the pins 16, 18 may optionally extend completely through the implant. Likewise, both the first 32, 34 and second 36, 38 ends of the pins 16, 18 can be buried in the implant. The pins 16, 18 are shown entering the implant from the same side 24, however they can enter from opposite sides 24, 26. Additional pins (not shown), for example third and fourth pins, similar to the first 16 and second 18 pins, can be positioned opposite the first and second pins 16, 18. FIG. 4 illustrates how the taper of the pins facilitates pinning from opposite sides as it allows them to occupy a smaller space 40 than non-tapered pins. The taper allows the pins to be placed closer together while maintaining a predetermined minimum spacing between the pins. Each pin has a longitudinal axis 37 and a diameter 39 associated with the larger end. The taper permits the axes 37 of the pins to cross one another in the body at an axial spacing 41 less than one-half the sum of the diameters 35 of the larger ends such that one pin passes through the envelope of the other.

The pins 16, 18 may optionally be chamfered 25, 27 at one or both ends to relieve stresses in the bone surrounding the ends of the pins 16, 18 and to relieve stresses in the pins themselves. When the pins 16, 18 are inserted into the bone pieces 12, 14, stresses may be generated that could lead to flaking of bone or fracturing of the bone pieces 12, 14 and/or pins 16, 18. Stresses may also be generated by subjecting the implant 10 to processes such as cleaning, drying, freezing, rehydrating, or other processes that may differentially affect the bone pieces 12, 14 and pins 16, 18. Likewise, the bone pieces may be chamfered 29 where the pins 16, 18 cross a surface 24. In the illustrative embodiment, a chamfer 29 is shown on surface 24, of the first bone piece 12. Chamfers may similarly be formed where the pins 16, 18 exit the first bone piece 12, where they enter and exit the third 50 and fourth 52 bone pieces, and where they enter the second bone piece 14.

Although the exemplary embodiment depicts the first and second bone pieces 12, 14 and pins 16, 18 as cortical bone, they may be any combination of bone material. The pins and bone pieces may be the same or different types of bones. Likewise, the pins may differ from one another and/or the pieces may differ from one another.

An implant comprising more than two bone pieces may be assembled according to the invention. Optional third 50 and fourth 52 bone pieces are shown positioned between the first 12 and second 14 bone pieces. In the example, the third 50 and fourth 52 bone pieces comprise cancellous bone to facilitate fusion of the adjacent bony structures. An optional opening 54 communicating between the adjacent bony structures further facilitates fusion. The illustrative implant 10 is positioned with two surfaces 20, 22 opposing adjacent bony structures to maintain the bony structures in a desired spaced relationship. The opening 54 extends between the two surfaces 20, 22 to provide a path for bone growth between the adjacent bony structures. The implant may further incorporate a bone growth promoting substance within the body and/or opening 54.

The implant components may be further interconnected by additional mechanical or chemical mechanisms, e.g. suturing, pressing, incorporating a binding agent, collagen crosslinking, entangling, and other suitable means and combinations thereof.

If the pieces are sutured together, holes may be formed in the pieces and a flexible, elongate, biocompatible connector may be threaded through the holes to interconnect the pieces. The connector may be a suture and/or elongate pieces of body tissue. Examples of materials for such connectors include pericardium, demineralized bone, fascia, cartilage, tendon, ligament, skin, collagen, elastin, reticulum, intestinal submucosa, metal, resorbable polymer, and nonresorbable polymer, and/or other suitable material.

If a binding agent is used to interconnect the pieces, it may be an adhesive binding agent, a cementitious binding agent, and/or other suitable binding agent. Examples of adhesive binding agents include fibrin glue, cyanoacrylate, epoxy, polymethylmethacrylate, gelatin based adhesives, and other suitable adhesives and combinations thereof. Examples of cementitious binding agents include settable ceramics, calcium carbonate, calcium phosphate, plaster, and other suitable materials and combinations thereof.

If the pieces are interconnected by collagen cross-linking, bone pieces may be partially demineralized to expose collagen fibers which may then be crosslinked by application of heat, pressure, chemicals, and/or other suitable cross-linking means.

FIG. 5 depicts the fusion implant 10 of FIG. 1 being used in conjunction with a fixation device 62 to form a bone fixation system 64. In such a system 64, the fusion implant 10 is positioned between adjacent bony structures 66, 68 desired to be fused together. The fixation device 62 may include one or more anchor mechanisms 72, such as screws, pins, wires, and/or other mechanisms for attaching it to the adjacent bony structures 66, 68 to limit the relative motion between them. The fixation device 62 may substantially prevent all relative motion, or it may allow a predetermined amount of motion, such as to allow the implant 10 to remain in contact with the adjacent bony structures 66, 68 during the healing and fusion processes. Suitable examples of a fixation device 62 include plates, internal or external rod systems, cable systems, cerclage systems, screws, and other suitable devices and combinations thereof.

Cortical bone pieces used to assemble the implant 10 may have a predetermined layer thickness and geometry, measured radially from the longitudinal axis of the donor bone, less than a predetermined minimum wall thickness and geometry. For example, the predetermined layer thickness and geometry may be in the range of less than 2 mm thick in one embodiment, less than 1.8 mm thick in another embodiment, less than 1.5 mm thick in yet another embodiment, less than 1.0 mm thick in still another embodiment, and less than 0.5 mm thick in another embodiment. Further, for example, the predetermined minimum wall thickness and geometry may relate to a minimum acceptable thickness or geometry associated with forming an integral or assembled load bearing implant. The predetermined minimum cortical geometry may vary depending on the application. For example, a minimum geometry for use in the cervical spine may be substantially less than a minimum cortical geometry for the lumbar spine. For instance, a predetermined minimum wall thickness or geometry for integral or assembled cortical wedge cervical spine implant, such as may be formed from a fibula, may be 3.0 mm in one embodiment, 2.5 mm in another embodiment, 2.0 mm in yet another embodiment, and 1.8 mm in still another embodiment. On the other hand, a minimum cortical geometry for an integral or assembled lumbar implant may be 4.5 mm in one embodiment, 4.0 mm in another embodiment, and 3.5 mm in another embodiment.

Implants formed from a plurality of bone pieces may have a compressive strength, or load bearing capacity, in the range of 50N to 20,000N. For instance, embodiments may have compressive strength greater than 70N, or greater than 800N, or greater than 1000N, or greater than 1200N, or greater than 3000N, or greater than 5000N, or greater than 7000N, or greater than 10,000N, or greater than 12,000N, or greater than 15,000N, or greater than 17,000N. This compressive strength provides load-bearing capability greater than typical cancellous bone and up to that of typical cortical bone.

Although embodiments of implants and methods of making and using them have been described and illustrated in detail, it is to be understood that the same is intended by way of illustration and example only and is not to be taken by way of limitation. Accordingly, variations in and modifications to the implants and methods will be apparent to those of ordinary skill in the art, and the following claims are intended to cover all such modifications and equivalents.

1. A fusion implant for insertion between opposing bony structures in load bearing arrangement, the implant comprising:
a body having first and second pieces of bone assembled together to form a construct having exterior surfaces; and
at least one pin projecting into the first and second body pieces to hold them together, the pin having a first end and a second end, a portion of the pin tapering between the first and second ends.

2. The implant of claim 1 wherein the at least one pin is chamfered at least one of its first and second ends.

3. The implant of claim 1 wherein the pin extends from a first exterior surface partway toward a second exterior surface such that the pin is exposed at the first surface and stops short of the second surface.

4. The implant of claim 1 wherein the tapered portion of the pin tapers away from a first exterior surface toward a second exterior surface such that the pin has a larger portion that is near the first exterior surface and a smaller portion that is near the second exterior surface.

5. The implant of claim 4 further comprising a second pin with a second tapered portion, the second tapered portion tapering away from the second exterior surface toward the first exterior surface such that the second pin has a larger portion that is near the second exterior surface and a smaller portion that is near the first exterior surface.

6. The implant of claim 5 wherein each pin further has a longitudinal axis and a diameter associated with its larger end, the axes of the pins crossing one another within the body at a distance between the axes that is less than one-half the sum of the diameters of the larger ends of the pins such that one pin passes through the envelope of the other.

7. The implant of claim 1 wherein the pin extends through the body from one exterior surface to another.

8. The implant of claim 1 wherein the pin is embedded within the body, such that it is surrounded on all sides by the body.

9. The implant of claim 1 wherein the body further comprises a third piece of bone positioned between the first and second pieces, the first and second pieces comprising bone having a first load bearing capacity and the third piece comprising bone having a second load bearing capacity, the pin extending into each of the pieces to hold the construct together.

10. The implant of claim 1 wherein the body further comprises a third piece of bone sandwiched between the first and second pieces, the first and second pieces comprising relatively dense bone and the third piece comprising relatively porous bone, the pin extending into each of the pieces to hold the construct together.

11. The implant of claim 10 wherein the first and second pieces comprise cortical bone and the third piece comprises cancellous bone.

12. The implant of claim 11 wherein the pin comprises cortical bone.

13. The implant of claim 10 wherein the exterior surfaces include opposing sides for contacting the adjacent bony structures, the first, second, and third pieces being aligned side by side such that each piece spans the adjacent bony structures, the body further including an opening through the third piece communicating with the adjacent bony structures.

14. The implant of claim 10 wherein the exterior surfaces include opposing sides for contacting the adjacent bony structures, the body further comprising a fourth piece of bone comprising relatively porous bone, the four pieces of bone being aligned side by side such that each piece spans the adjacent bony structures, the third and fourth pieces being spaced apart to form an opening through the body communicating with the adjacent bony structures.

15. The implant of claim 1 further comprising an opening through the body communicating with the adjacent bony structures.

16. The implant of claim 15 further comprising a bone growth promoting substance in the opening.

17. The implant of claim 16 wherein the bone growth promoting substance comprises at least one substance selected from the group consisting of bone paste, cancellous bone, bone chips, bone morphogenic protein, LIM mineralization protein, platelet derived growth factors, bone marrow aspirate, stem cells, and biologic growth factors.

18. The implant of claim 1 wherein the pin comprises at least one material selected from the group consisting of bone, metal, ceramic, carbon, bioglass, and polymers.

19. The implant of claim 1 further comprising a second pin.

20. The implant of claim 1 wherein the pin angles through the construct obliquely such that it is neither parallel nor perpendicular to any of the exterior surfaces.

21. The implant of claim 1 further comprising a fixation device attached to the adjacent bony structures to limit the relative motion between them.

22. The implant of claim 21 wherein the fixation device substantially prevents all relative motion between the adjacent bony structures.

23. The implant of claim 21 wherein the fixation device allows a predetermined amount of relative motion between the adjacent boney structures during the fusion process.

24. The implant of claim 21 wherein the fixation device is selected from the group consisting of plates, internal rod systems, external rod systems, cable systems, cerclage systems, screws, and combinations thereof.

25. A fusion implant for insertion between opposing bony structures in load bearing arrangement, the implant comprising:
a body having first and second pieces of bone assembled together to form a construct; and
at least one pin projecting into the first and second body pieces to hold them together, the pin having a first dimension adjacent an end and a second dimension spaced from the end into the body.

26. The implant of claim 25 wherein the first dimension is greater than the second dimension.

27. The implant of claim 25 further comprising a fixation device attached to the adjacent bony structures to limit the relative motion between them.

28. A fusion implant for insertion between opposing bony structures in load bearing arrangement, the implant comprising:
a body having first and second pieces of bone assembled together to form a construct having exterior surfaces; and
a pin projecting into the first and second body pieces to hold them together, the pin angling through the construct obliquely such that it is neither parallel nor perpendicular to any of the exterior surfaces.

29. The implant of claim 28 further comprising a fixation device attached to the adjacent bony structures to limit the relative motion between them.

30. A fusion implant for insertion between opposing bony structures in load bearing arrangement, the implant comprising:
a body having first and second pieces of bone assembled together; and
a pin projecting into the first and second body pieces to hold them together, the pin being embedded within the body, such that it is surrounded on all sides by the body.

31. A fusion implant for insertion between opposing bony structures in load bearing arrangement, the implant comprising:
a body having first, second, and third pieces of bone assembled together, the pieces being aligned side-by-side with the second piece of bone positioned between the first and third pieces of bone such that each piece spans the adjacent bone structures, the body including an opening through the third piece communicating with the adjacent bony structures.

32. The implant of claim 31 further comprising a fusion promoting substance.

33. The implant of claim 31 further comprising a fixation device attached to the adjacent bony structures to limit the relative motion between them.

34. A fusion implant for insertion between opposing bony structures in load bearing arrangement, the implant comprising:
a body having first, second, third and fourth pieces of bone assembled together, the pieces being aligned side-by-side with the second and third pieces of bone positioned between the first and fourth pieces of bone such that each piece spans the adjacent bony structures, the second and third pieces being spaced apart to form an opening through the body communicating with the adjacent bony structures.

35. A system for use in fusing adjacent bony structures, comprising:
a body having first and second pieces of bone assembled together to form a construct having exterior surfaces;
a pin projecting into the first and second body pieces to hold them together, the pin having a first end and a second end, the portion of the pin tapering between the first and second ends; and
a fixation device attachable to the adjacent bony structures and having a structure to limit relative motion between the adjacent bony structures.

36. A system for use in fusing adjacent bony structures, comprising:
a body having first and second pieces of bone assembled together to form a construct having exterior surfaces;
a pin projecting into the first and second body pieces to hold them together, the pin angling through the construct obliquely such that it is neither parallel nor perpendicular to any of the exterior surfaces; and
a fixation device attachable to the adjacent bony structures and having a structure to limit relative motion between the adjacent bony structures.

37. A system for use in fusing adjacent bony structures, comprising:
a body having first, second, and third pieces of bone assembled together, the pieces being aligned side-by-side with the second piece of bone positioned between the first and third pieces of bone such that each piece spans the adjacent bone structures, the body including an opening through the third piece communicating with the adjacent bony structures; and
a fixation device attachable to the adjacent bony structures and having a structure to limit relative motion between the adjacent bony structures.

38. A method of treating adjacent bony structures comprising:
providing a body having first and second pieces of bone assembled together to form a construct having exterior surfaces, and a pin projecting into the first and second body pieces to hold them together, the pin having a first end and a second end, a portion of the pin tapering between the first and second ends; and
positioning the implant between the adjacent bony structures in load bearing arrangement.

39. A method of treating adjacent bony structures comprising:
providing a body having first and second pieces of bone assembled together to form a construct having exterior surfaces, and a pin projecting into the first and second body pieces to hold them together, the pin angling through the construct obliquely such that it is neither parallel nor perpendicular to any of the exterior surfaces; and
positioning the implant between the adjacent bony structures in load bearing arrangement.

40. A method of treating adjacent bony structures comprising:
providing a body having first, second, and third pieces of bone assembled together, the pieces being aligned side-by-side with the second piece of bone positioned between the first and third pieces of bone such that each piece spans the adjacent bone structures, the body including an opening through the third piece communicating with the adjacent bony structures; and
positioning the implant between the adjacent bony structures in load bearing arrangement.

41. A method of making a bone implant comprising:
forming first and second bone pieces;
assembling the first and second pieces to form a body; and
inserting a pin into the body to hold the assembly together, the pin having a first end and a second end, a portion of the pin tapering between the first and second ends.

42. A method of making a bone implant comprising:
forming first and second bone pieces;
assembling the first and second pieces to form a body having exterior surfaces; and
inserting a pin into the body to hold the assembly together, the pin angling through the construct obliquely such that it is neither parallel nor perpendicular to any of the exterior surfaces.
